(19) United States
(12) Patent Application Publication
(10) Pub. No.: US 2014/0193082 A1
(43) Pub. Date: Jul. 10, 2014

(54) IMAGE PROCESSING APPARATUS AND METHOD

(71) Applicants: Toshiba Medical Systems Corporation, Otawara-shi (JP); KABUSHIKI KAISHA TOSHIBA, Minato-ku (JP)

(72) Inventors: Kae OHNUKI, Yokohama-shi (JP); Hisanori KATO, Otawara-shi (JP); Yasuhiro SUGAWARA, Nasushiobara-shi (JP)

(73) Assignees: Toshiba Medical Systems Corporation, Otawara-shi (JP); KABUSHIKI KAISHA TOSHIBA, Minato-ku (JP)

(21) Appl. No.: 14/205,544

(22) Filed: Mar. 12, 2014

Related U.S. Application Data
(63) Continuation of application No. PCT/JP2012/079680, filed on Nov. 15, 2012.

(30) Foreign Application Priority Data
Nov. 15, 2011 (JP) 2011-250066
Nov. 15, 2012 (JP) 2012-251081

(51) Int. Cl. G06T 5/50; G06K 9/00

(52) U.S. Cl. CPC G06T 5/50; G06K 9/00523
USPC 382/205

(57) ABSTRACT

According to one embodiment, an image processing apparatus includes following units. The selection unit selects a pixel from a target image. The first extraction unit extracts a first pixel region including the selected pixel. The second extraction unit extracts a second pixel region from a reference image. The determination unit determines a filter coefficient based on a similarity degree between the first and second pixel regions. The generation unit generates a display image by performing a weighted sum of the target image and a display image generated immediately before the target image, in accordance with the filter coefficient determined for each of the plurality of pixels of the target image.

### CROSS REFERENCE TO RELATED APPLICATIONS

This application is a Continuation Application of PCT Application No. PCT/JP2012/079680, filed Nov. 15, 2012 and based upon and claiming the benefit of priority from Japanese Patent Applications No. 2011-250066, filed Nov. 15, 2011; and No. 2012-251081, filed Nov. 15, 2012, the entire contents of all of which are incorporated herein by reference.

### FIELD

Embodiments described herein relate generally to an X-ray diagnostic apparatus including an image processing apparatus.

### BACKGROUND

As a medical technique using an X-ray diagnostic apparatus, for example, catheter treatment is performed under fluoroscopy. In X-ray fluoroscopy, since the dose of X-rays is reduced to reduce the exposure amounts for an object to be examined and a medical technician, large noise is superimposed on an image. The X-ray diagnostic apparatus includes an image processing apparatus which performs filter processing by using a recursive filter to reduce noise in an image. A recursive filter is a filter which performs weighted sum of a plurality of temporally continuous images in accordance with filter coefficients. Conventionally, each filter coefficient is set to a constant value within an image.

The recursive filter has, however, the problem that a residual image occurs on a portion where an object such as a catheter or an organ of an object to be examined moves, resulting in motion blur of a displayed image.

### BRIEF DESCRIPTION OF THE DRAWINGS

FIG. 1 is a block diagram schematically showing an X-ray diagnostic apparatus according to the first embodiment;

FIG. 2 is a block diagram schematically showing an image processing unit shown in FIG. 1;

FIG. 3 is a schematic view showing the X-ray images captured by an imaging unit shown in FIG. 1;

FIG. 4 is a schematic view showing an example of the order in which a selection unit shown in FIG. 2 selects pixels;

FIG. 5 is a flowchart showing an example of the operation of the image processing unit in FIG. 2;

FIG. 6 is a graph schematically showing the data held by a reference table stored in a filter coefficient determination unit shown in FIG. 2;

FIG. 7 is a schematic view showing an example of the coefficient map generated by a filter coefficient storage unit shown in FIG. 2;

FIG. 8 is a block diagram schematically showing an image processing unit according to the second embodiment;

FIG. 9 is a flowchart showing an example of the operation of the image processing unit in FIG. 8; and

FIG. 10 is a schematic view showing X-ray images input to the image processing unit in FIG. 8.

### DETAILED DESCRIPTION

In general, according to one embodiment, an image processing apparatus includes a first storage unit, a selection unit, a first extraction unit, a second extraction unit, a calculation unit, a determination unit, and a generation unit. The first storage unit is configured to store data of a plurality of images. The selection unit is configured to select a pixel from a plurality of pixels included in a target image of the plurality of images. The first extraction unit is configured to extract a first pixel region including the selected pixel from the target image. The second extraction unit is configured to extract a second pixel region corresponding to the first pixel region from a reference image of the plurality of images, the reference image being different from the target image. The calculation unit is configured to calculate a similarity degree between the first pixel region and the second pixel region. The determination unit is configured to determine a filter coefficient based on the similarity degree. The generation unit is configured to generate a display image by performing a weighted sum of the target image and a display image generated immediately before the target image, in accordance with the filter coefficient determined for each of the plurality of pixels.

An image processing apparatus and method according to an embodiment will be described below with reference to the accompanying drawings. The embodiment will exemplify an X-ray diagnostic apparatus including an image processing apparatus. In the embodiments, like reference numbers denote like elements, and a repetitive description of them will be omitted.

### First Embodiment

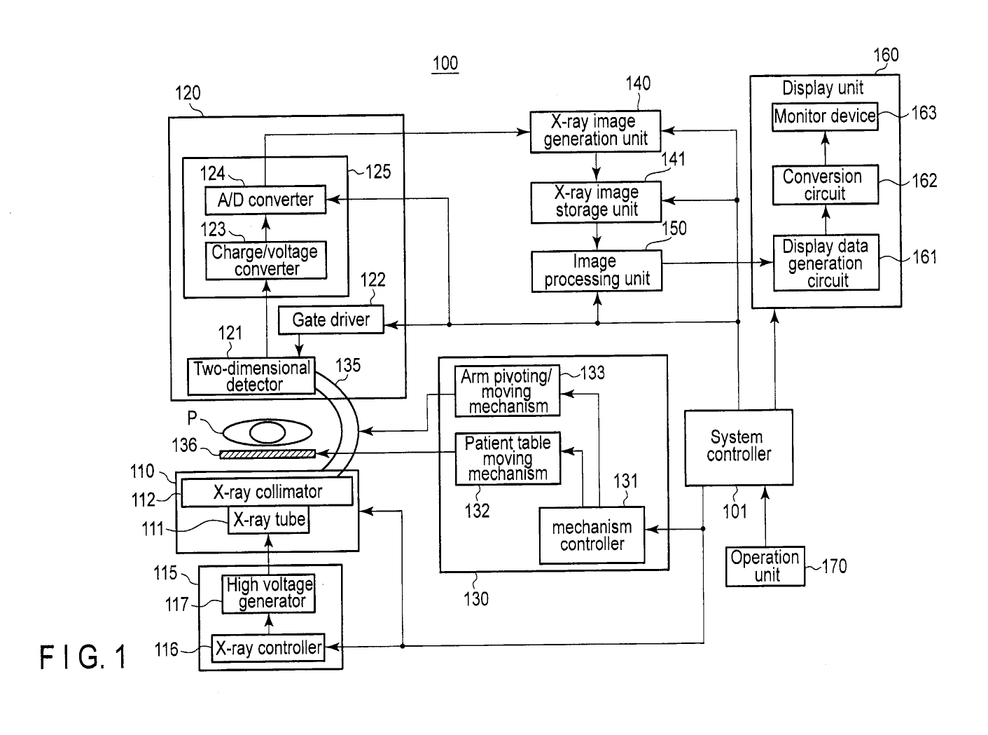

FIG. 1 schematically shows an X-ray diagnostic apparatus 100 according to the first embodiment. As shown in FIG. 1, the X-ray diagnostic apparatus 100 includes a C-arm 135 in the form of the letter C. The C-arm 135 is supported by an arm support portion (not shown) so as to be pivotal and movable. An X-ray generation unit 110 which generates X-rays is provided on one end of the C-arm 135. An X-ray detection unit 120 which detects the X-rays emitted from the X-ray generation unit 110 and transmitted through an object P is provided on the other end of the C-arm 135. The X-ray generation unit 110 and the X-ray detection unit 120 are arranged to face each other through the object P placed on a patient table 136 provided on a bed device (not shown). An operation unit 170 is provided on the bed device.

A mechanical unit 130 positions the C-arm 135 and the patient table 136. The mechanical unit 130 includes a mechanism controller 131, a patient table moving mechanism 132, and an arm pivoting/moving mechanism 133. The mechanism controller 131 generates driving signals for driving the patient table moving mechanism 132 and the arm pivoting/moving mechanism 133 in accordance with movement control commands from a system controller 101. The patient table moving mechanism 132 moves the patient table 136 by being driven by a driving signal from the mechanism controller 131. The arm pivoting/moving mechanism 133 is driven by a driving signal from the mechanism controller 131 to move the C-arm 135 and cause the C-arm 135 to pivot about the body axis of the object P. Adjusting the position of the patient table 136 and the position and angle of the C-arm 135 in this manner will adjust the positions of the X-ray generation unit 110 and X-ray detection unit 120 relative to the object P.

A high voltage generation unit 115 is connected to the X-ray generation unit 110. The high voltage generation unit 115 applies a high voltage to the X-ray generation unit 110. More specifically, the X-ray generation unit 110 includes an X-ray controller 116 and a high voltage generator 117. The X-ray controller 116 receives an X-ray irradiation command including X-ray conditions from the system controller 101, generates a voltage application control signal for generating the voltage designated by the X-ray conditions, and sends out the signal to the high voltage generator 117. For example, the X-ray conditions include a tube voltage to be applied between the electrodes of an X-ray tube 111 of the X-ray generation unit 110, a tube current, an X-ray irradiation time, and an X-ray irradiation timing. The high voltage generator 117 generates a high voltage in accordance with the voltage application control signal received from the X-ray controller 116 and applies the voltage to the X-ray generation unit 110.

The X-ray generation unit 110 includes the X-ray tube 111 and an X-ray collimator 112. The high voltage generator 117 applies a high voltage to the X-ray tube 111 to make it generate X-rays. The X-ray collimator 112 is disposed between the X-ray tube 111 and the object P to limit the irradiation field of X-rays emitted from the X-ray tube 111 to the object P.

The X-ray detection unit 120 includes a two-dimensional detector 121, a gate driver 122, and a projection data generation unit 125. The two-dimensional detector 121 includes a plurality of semiconductor detection elements arrayed two-dimensionally. The gate driver 122 generates a driving pulse for reading out charges accumulated in the two-dimensional detector 121. The X-rays transmitted through the object P are converted into charges and accumulated by the semiconductor detection elements of the two-dimensional detector 121. The accumulated charges are sequentially read out by the driving pulses supplied from the gate driver 122.

The projection data generation unit 125 converts the charges read out from the two-dimensional detector 121 into projection data. More specifically, the projection data generation unit 125 includes a charge/voltage converter 123 and an A/D converter 124. The charge/voltage converter 123 converts each charge read out from the two-dimensional detector 121 into a voltage signal. The A/D converter 124 converts the voltage signal output from the charge/voltage converter 123 into a digital signal and outputs it as projection data.

An X-ray image generation unit 140 generates an X-ray image (fluoroscopic image) based on the projection data output from the projection data generation unit 125, and stores the generated X-ray image in an X-ray image storage unit 141. In this embodiment, the X-ray generation unit 110 continuously emits X-rays to the object P. The X-ray detection unit 120 executes X-ray detection at a predetermined period (e.g., a period of 1/30 sec) to acquire a plurality of X-ray images concerning the object P in chronological order. That is, an X-ray moving image of the object P is captured. An X-ray moving image includes X-ray images corresponding to several ten frames per sec. The X-ray image storage unit 141 stores captured X-ray images together with frame numbers indicating the times (or ordinal numbers) at which the respective X-ray images have been captured. An imaging unit which captures X-ray moving images is formed by the X-ray generation unit 110, the high voltage generation unit 115, the X-ray detection unit 120, the mechanical unit 130, the C-arm 135, the patient table 136, the X-ray image generation unit 140, and the X-ray image storage unit 141.

The X-ray diagnostic apparatus 100 further includes an image processing unit 150. The image processing unit 150 generates a display image by performing recursive filter processing (to be described later) for an X-ray image stored in the X-ray image storage unit 141. The display image generated by the image processing unit 150 is sent to a display unit 160.

The display unit 160 displays the display image generated by the image processing unit 150. More specifically, the display unit 160 includes a display data generation circuit 161, a conversion circuit 162, and a monitor device 163. The display data generation circuit 161 receives a display image from the image processing unit 150 and generates display data to be displayed by the monitor device 163. The conversion circuit 162 converts the display data generated by the display data generation circuit 161 into a video signal and sends it out to the monitor device 163. As a result, the monitor device 163 displays an X-ray image of the object P. As the monitor device 163, a CRT (Cathode-Ray Tube) display, an LCD (Liquid Crystal Display), or the like can be used.

The operation unit 170 includes input devices such as a keyboard and a mouse. The operation unit 170 accepts an input from the user, generates an operation signal corresponding to the input, and sends out the signal to the system controller 101. For example, the operation unit 170 is used to set X-ray conditions.

The system controller 101 controls the overall X-ray diagnostic apparatus 100. For example, the system controller 101 controls the imaging unit, the image processing unit 150, and the display unit 160 to capture an X-ray moving image of an object and display the image in real time. When capturing an X-ray moving image, the system controller 101 performs adjustment of an X-ray dose, ON/OFF control of X-ray irradiation, and the like in accordance with the X-ray conditions input from the operation unit 170.

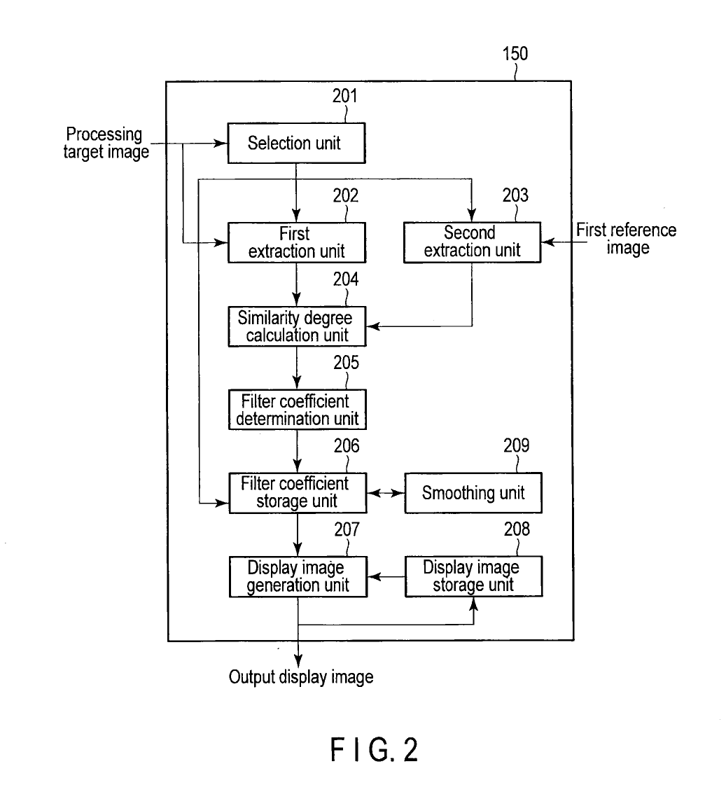

FIG. 2 schematically shows the image processing unit 150 according to this embodiment. As shown in FIG. 2, the image processing unit 150 includes a selection unit 201, a first extraction unit 202, a second extraction unit 203, a similarity degree calculation unit 204, a filter coefficient determination unit 205, a filter coefficient storage unit 206, a display image generation unit 207, and a display image storage unit 208. The image processing unit 150 sequentially receives X-ray images stored in the X-ray image storage unit 141 shown in FIG. 1 in accordance with a frame order. The image processing unit 150 may include the X-ray image storage unit 141.

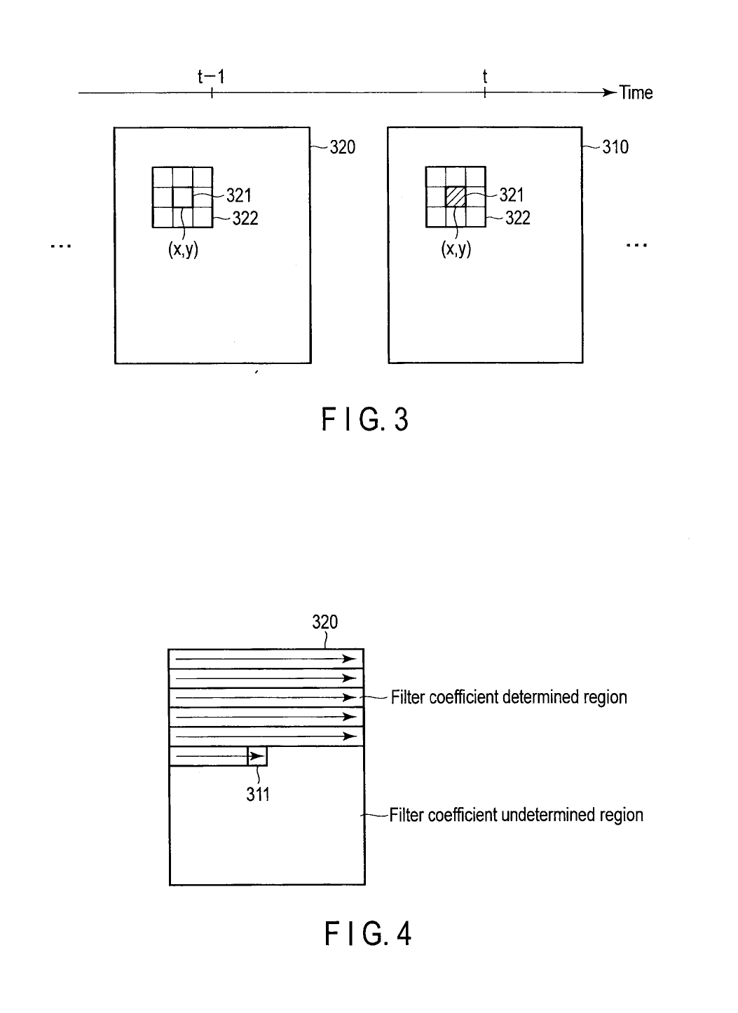

In the image processing unit 150, X-ray images acquired in chronological order are sequentially sent to the selection unit 201 and the first extraction unit 202. A one-frame X-ray image sent as a recursive filter processing target to the selection unit 201 and the first extraction unit 202 will be referred to as a target image hereinafter. An X-ray image one frame before the target image is sent as the first reference image to the second extraction unit 203. For example, as shown in FIG. 3, the target image is an X-ray image 310 at time t, and the first reference image is an X-ray image 320 at time t−1.

The selection unit 201 sequentially selects a pixel 311 from a plurality of pixels included in the target image 310. Position information indicating the position of the selected pixel 311 is sent to the first extraction unit 202, the second extraction unit 203, and the filter coefficient storage unit 206. As shown in FIG. 4, the pixels in the target image 310 are selected one by one in, for example, a raster scan order. Note that the selection order is not limited to the raster scan order, and may be any order.

As shown in FIG. 3, the first extraction unit 202 extracts, from the target image 310, a pixel block 312 including the pixel 311 specified by the position information from the selection unit 201. Referring to FIG. 3, the pixel 311 selected by the selection unit 201 is indicated by the hatching. The pixel block 312 in this embodiment is formed by the pixel 311 and eight pixels adjacent to the pixel 311. That is, the pixel block 312 is a 3×3 pixel block with the selected pixel 311 being placed in the center. Note that the pixel block 312 is not limited to the square pixel block shown in FIG. 3 and may have an arbitrary shape. In addition, the selected pixel 311 may be placed in the center of the pixel block 312.

The second extraction unit 203 extracts a pixel block 322 corresponding to the pixel block 312 from the reference image 320. The pixel block 322 in this embodiment is a pixel block having the same size as that of the first pixel block 312, and includes a pixel 321 specified by the position information from the selection unit 201. More specifically, the pixel block 322 is a 3×3 pixel block with the pixel 321 being placed in the center.

The similarity degree calculation unit 204 calculates the similarity degree between the pixel block 312 extracted from the X-ray image 310 and the pixel block 322 extracted from the reference image 320. The filter coefficient determination unit 205 determines a filter coefficient (weighting coefficient) concerning the selected pixel 311 based on the similarity degree calculated by the similarity degree calculation unit 204. The filter coefficient storage unit 206 stores the filter coefficient determined concerning the selected pixel 311 in correspondence with the position information. The image processing unit 150 sequentially selects the pixels in the target image 310. As a result, a filter coefficient is determined concerning each pixel in the target image 310.

The display image generation unit 207 generates a display image by performing a weighted sum of the target image 310 and the second reference image stored in the display image storage unit 208 in accordance with the filter coefficients stored in the filter coefficient storage unit 206. The display image generated when an X-ray image at time t is set as the target image 310 is a display image at time t. When a display image at time t is generated, a display image at time t−1 generated immediately before is stored as the second referenced image in the display image storage unit 208. The display image at time t generated by the display image generation unit 207 is sent to the display unit 160 and is stored as the new second reference image in the display image storage unit 208 to be used for the generation of a display image at next time t+1. Recursively using generated display images in this manner can effectively remove noise randomly generated in an X-ray image.

The image processing unit 150 may be provided with a smoothing unit 209 which smoothes the filter coefficients determined concerning the pixels in the target image 310. If the image processing unit 150 is provided with the smoothing unit 209, the display image generation unit 207 generates a display image by using the filter coefficients smoothed by the smoothing unit 209. Smoothing the filter coefficient determined for each pixel can generate a more natural display image.

The operation of the X-ray diagnostic apparatus 100 will be described next.

A method by which the imaging unit acquires X-ray images will be briefly described first.

The object P is placed on the patient table 136 of the bed. Upon receiving a movement control command from the system controller 101, the mechanism controller 131 sends out driving signals to the patient table moving mechanism 132 and the arm pivoting/moving mechanism 133, respectively. The patient table moving mechanism 132 is activated by a driving signal to adjust the patient table 136 to a desired position. In addition, the arm pivoting/moving mechanism 133 is activated by a driving signal to adjust the C-arm 135 to a desired position and angle.

The system controller 101 further sends out X-ray irradiation commands including X-ray conditions to the X-ray controller 116 and the X-ray generation unit 110. With this operation, the X-ray controller 116 generates a voltage application control signal for generating the voltage designated by X-ray conditions and sends out the signal to the high voltage generator 117. The high voltage generator 117 generates a high voltage corresponding to the voltage application control signal from the X-ray controller 116 and applies the voltage to the X-ray generation unit 110. When a high voltage is applied to the X-ray tube 111 of the X-ray generation unit 110, the X-ray tube 111 generates X-rays and emits them to the object P.

The X-rays emitted from the X-ray tube 111 pass through the X-ray collimator 112 and enter the two-dimensional detector 121 through the object P. The semiconductor detection elements convert the X-rays which have entered the two-dimensional detector 121 into charges, which are then accumulated in the semiconductor detection elements. The accumulated charges are read out by driving pulses from the gate driver 122. The charge/voltage converter 123 converts the read out charges into voltage signals. The A/D converter 124 converts the voltage signals from the charge/voltage converter 123 into digital signals and outputs them as projection data. The X-ray image generation unit 140 generates X-ray images concerning the object P in chronological order based on the projection data.

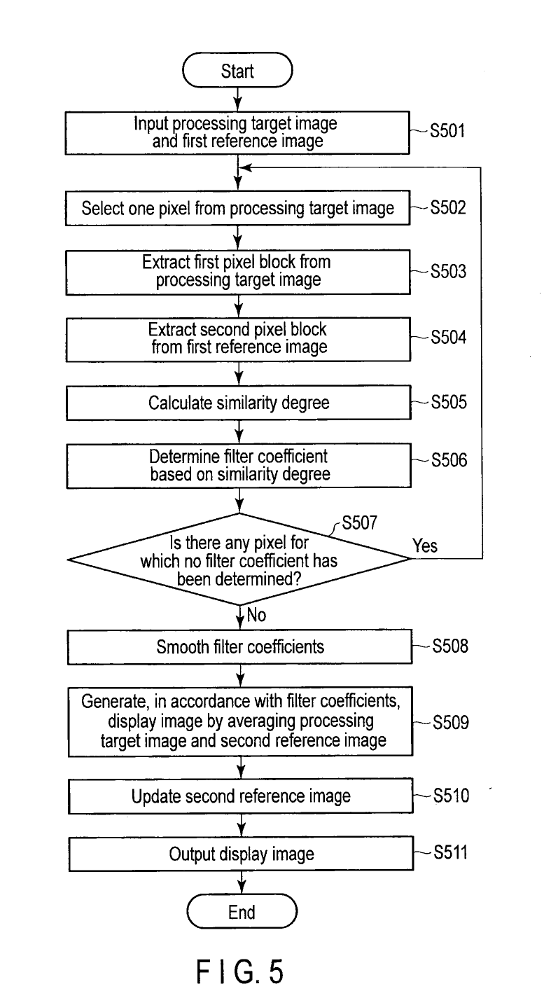

An example of recursive filter processing by the image processing unit 150 will be described next with reference to FIG. 5.

In step S501 in FIG. 5, the image processing unit 150 receives an X-ray image at a given time as a target image and an X-ray image one frame before the target image as the first reference image. The following is a case in which the target image is the X-ray image 310 at time t, and the first reference image is the X-ray image 320 at time t−1, as shown in FIG. 3.

In step S502, the selection unit 201 selects a pixel 311 from the target image 310. Assume that in this embodiment, the position of each pixel in an X-ray image is represented by a coordinate (x, y), and a pixel is placed at a position where components x and y of the coordinate (x, y) are integer values. Assume that the position of the pixel 311 selected by the selection unit 201 in step S502 is represented by the coordinate (x, y).

In step S503, the first extraction unit 202 extracts the first pixel block 312 including the pixel 311 selected in step S502 from the target image 310. The first pixel block 312 in this embodiment is a 3×3 pixel block with the selected pixel 311 being placed in the center.

In step S504, the second extraction unit 203 extracts the second pixel block 322 corresponding to the first pixel block 312 extracted in step S503 from the first reference image 320. The second pixel block 322 in this embodiment is a pixel block on the first reference image 320, that is, a 3×3 pixel block with the pixel 321 located at the same coordinate (x, y) as those of the selected pixel 311 being placed in the center.

In step S505, the similarity degree calculation unit 204 calculates the similarity degree between the first pixel block 312 and the second pixel block 322. For example, the similarity degree calculation unit 204 calculates a similarity degree S(x, y) based on the difference value between the pixel value of the first pixel block 312 and the pixel value of the second pixel block 322 as indicated by equation (1):

$$S ( x , y ) = A \times exp ( - B \times {\sum }_{{- 1 \leq i \leq 1}_{- 1 \leq j \leq 1}} \vert {I}_{t} ( x + i , y + j ) - {I}_{t - 1} ( x + i , y + j ) \vert ) \qquad ( 1 )$$

where Iₜ(x, y) represents the pixel value of a pixel at the coordinate (x, y) on the target image 310, and Iₜ₋₁(x, y) represents the pixel value of a pixel at the coordinate (x, y) on the first reference image 320. Since an X-ray image is a monochrome image, each pixel of the X-ray image has a luminance value as a pixel value. That is, a pixel value Iₜ(x, y) and a pixel value Iₜ₋₁(x, y) are scalar values. In addition, in equation (1), A and B are predetermined positive values.

As indicated by equation (1), the similarity degree S(x, y) increases as the first pixel block 312 is similar to the second pixel block 322. That is, the similarity degree S(x, y) is high in a still region where a change in pixel value between frames is small, whereas the similarity degree S(x, y) is low in a dynamic region where a change in pixel value between frames is large.

The calculation of the similarity degree S(x, y) is not limited to that based on equation (1), and may be performed according to another calculation formula. For example, the similarity degree S(x, y) may be based on the square sum of the differences between pixel values. In the above case, pixel values are scalar values. However, pixel values may be vectors as in a case in which color images are handled.

In step S506, the filter coefficient determination unit 205 determines a filter coefficient G(x, y) based on the similarity degree S(x, y) calculated by the similarity degree calculation unit 204. For example, the filter coefficient determination unit 205 stores a reference table holding data concerning a plurality of similarity degrees together with data concerning filter coefficients respectively associated with the plurality of similarity degrees. The filter coefficient determination unit 205 refers to the reference table with the similarity degree S(x, y) calculated by the similarity degree calculation unit 204 to acquire the filter coefficients G(x, y) associated with the similarity degree S(x, y). In another example, the filter coefficient determination unit 205 may hold the relationship between similarity degrees and filter coefficients in a functional form.

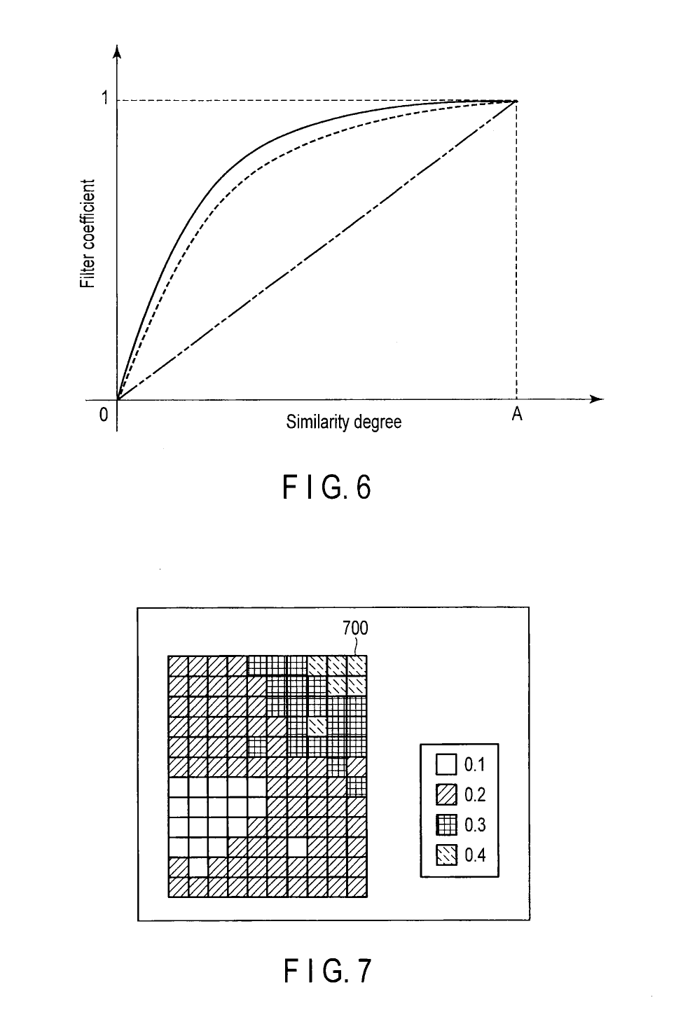

FIG. 6 is a graph showing the data held in the reference table of the similarity degree calculation unit 204. As indicated by the solid line in FIG. 6, the filter coefficients G(x, y) in this embodiment takes a value equal to or more than 0 and equal to or less than 1, and increases with an increase in the similarity degree S(x, y). If, therefore, the selected pixel 311 falls within a still region, the filter coefficients G(x, y) obtained is large. In contrast to this, if the selected pixel 311 falls within a dynamic region, the filter coefficients G(x, y) obtained is small. The filter coefficient storage unit 206 stores the determined filter coefficients G(x, y) in correspondence with the position information of the selected pixel 311.

Note that the relationship between similarity degrees and filter coefficients may be changed in accordance with X-ray conditions, as indicated by the broken line or two-dot dashed line in FIG. 6. The relationship between similarity degrees and filter coefficients in this case may be set to approach the two-dot dashed line with an increase in X-ray dose in FIG. 6. That is, if similarity degrees and filter coefficients have, for example, the relationship indicated by the solid line when the X-ray dose is α, the relationship between similarity degrees and filter coefficients is indicated by the broken line or two-dot dashed line if the X-ray dose is β (β>α). The relationship between similarity degrees and filter coefficients may be automatically changed to suitable conditions when the X-ray conditions have changed or may be changed as the operator operates the operation unit 170. In either case, the filter coefficients G(x, y) increases with an increase in the similarity degree S(x, y).

In step S507, the image processing unit 150 determines whether filter coefficients have been determined for all the pixels in the target image 310. If there is any pixel for which no filter coefficient has been determined, the process returns to step S502. The image processing unit 150 repeats the processing from step S502 to step S506 until filter coefficients are determined for all the pixels in the target image 310.

If the image processing unit 150 has determined filter coefficients for all the pixels in the target image 310, the process advances to step S508. In step S508, the smoothing unit 209 smoothes the filter coefficients determined for the respective pixels. The filter coefficient storage unit 206 stores the filter coefficients in association with position information. As shown in FIG. 7, the smoothing unit 209 generates a coefficient map (filter coefficient image) with filter coefficients being placed at pixel positions in accordance with position information. The smoothing unit 209 then smoothes the filter coefficients by using, for example, an averaging filter or Gaussian filter.

In step S509, the display image generation unit 207 generates a display image at time t corresponding to the target image 310 by using the filter coefficient determined for each pixel in the target image 310. For example, the display image generation unit 207 calculates, for each pixel, a pixel value I_{t′}(x, y) of the display image at time t by performing a weighted sum of the pixel value Iₜ(x, y) of the target image 310 and a pixel value I_{t-1′}(x, y) of the second reference image stored in the display image storage unit 208 by using the filter coefficients G(x, y) according to equation (2). The second reference image is the display image at time t−1 generated immediately before.

Iₜ^{′}(x,y)=Iₜ₋₁^{′}(x,y)×G(x,y)+Iₜ(x,y)×(1−G(x,y)) (2)

As indicated by equation (2), the influence of the second reference image on the display image increases with an increase in filter coefficient. As described above, large filter coefficients are determined concerning pixels in a still region, whereas small filter coefficients G are determined concerning pixels in a dynamic region. Therefore, in the still region, the influence of the second reference image is large, and it is possible to reduce noise. In the dynamic region, the influence of the second reference image is small, and it is possible to suppress the occurrence of a residual image. This makes it possible to generate a display image with less residual image and reduced noise.

In step S510, the display image storage unit 208 temporarily stores the generated display image as the new second reference image. In step S511, the generated display image is output to the display unit 160. Performing recursive filter processing in this manner can generate a display image with less residual image and reduced noise. This makes it possible to display a clear moving image without motion blur.

Although the above description has exemplified the case in which an X-ray image one frame before the target image is used as the first reference image, a plurality of X-ray images before the target image may be used as the first reference images.

As described above, since the X-ray diagnostic apparatus 100 according to this embodiment includes the image processing unit which determines a filter coefficient for each pixel in an X-ray image, it is possible to display an X-ray image with less motion blur and reduced noise.

### Second Embodiment

The second embodiment differs from the first embodiment in the arrangement of an image processing unit. In the first embodiment, the image processing unit extracts one second pixel block from the first reference image and determines filter coefficients based on the second pixel block. In contrast to this, in the second embodiment, the image processing unit extracts a plurality of second pixel blocks from the first reference image, calculates the similarity degrees between the first pixel block and the respective second pixel blocks, detects the second pixel block exhibiting the highest similarity degree, and determines filter coefficients based on the detected second pixel block.

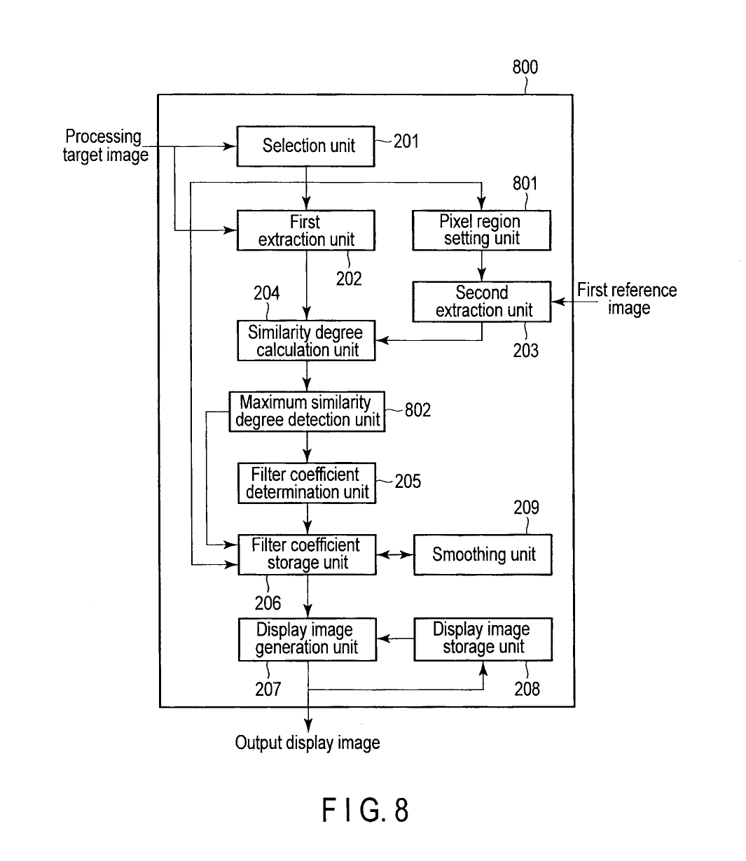

FIG. 8 schematically shows an image processing unit 800 according to the second embodiment. The image processing unit 800 shown in FIG. 8 includes a pixel region setting unit 801 and a maximum similarity degree detection unit 802 in addition to the arrangement of the image processing unit 150 shown in FIG. 2. The pixel region setting unit 801 sets a pixel region for the extraction of the second pixel blocks on the first reference image. The maximum similarity degree detection unit 802 detects the maximum similarity degree among the similarity degrees determined by a similarity degree calculation unit 204.

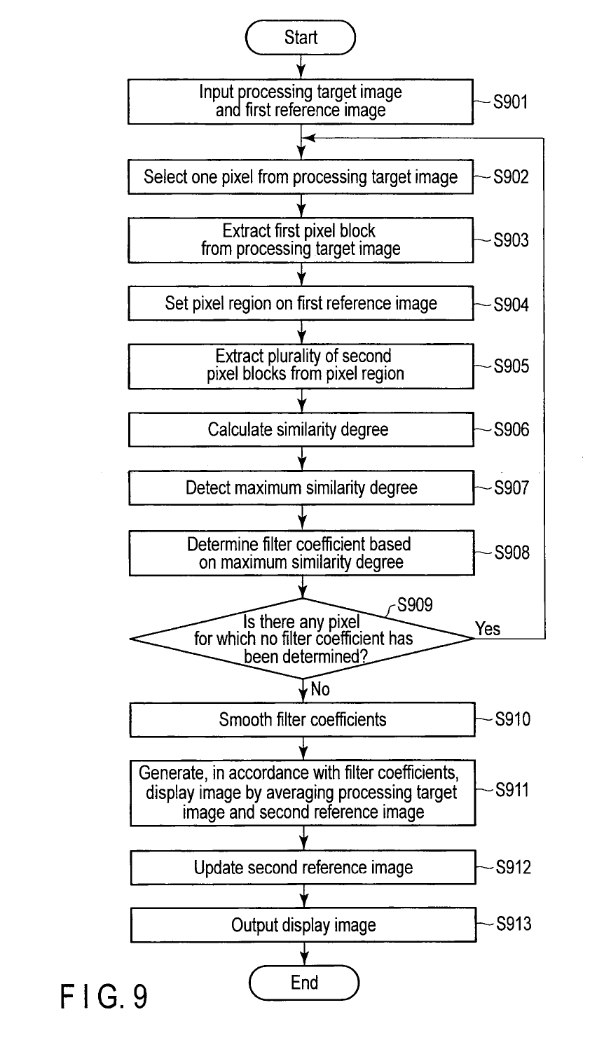

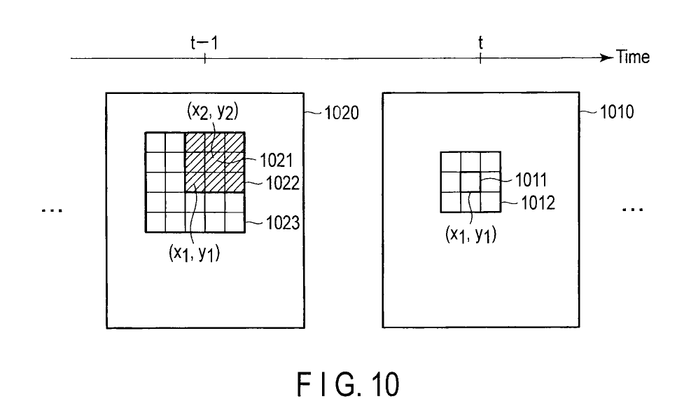

FIG. 9 shows an example of the operation of the image processing unit 800. In step S901 in FIG. 9, the image processing unit 800 receives an X-ray image at a given time as a target image and an X-ray image one frame before the target image as the first reference image. In this case, as shown in FIG. 10, assume that the target image is an X-ray image 1010 at time t, and the first reference image is an X-ray image 1020 at time t−1.

In step S902, a selection unit 201 selects a pixel 1011 from the target image 1010. The coordinate of the selected pixel 1011 is represented by a coordinate (x1, y1). Position information indicating the coordinate (x1, y1) of the selected pixel 1011 is sent to a first extraction unit 202, a filter coefficient storage unit 206, and the pixel region setting unit 801.

In step S903, the first extraction unit 202 extracts a first pixel block 1012 including the pixel 1011 selected in step S902 from the target image 1010. The first pixel block 1012 in this embodiment is a 3×3 pixel block with the selected pixel 1011 being placed in the center.

In step S904, the pixel region setting unit 801 sets a pixel region 1023 having a predetermined size on the first reference image 1020 in accordance with the position information from the selection unit 201. In the case shown in FIG. 10, the pixel region 1023 is a 5×5 pixel region centered on a pixel on the first reference image 1020 which is specified by the position information from the selection unit 201. The pixel region 1023 may have any size larger than that of the first pixel block 1010.

In step S905, a second extraction unit 203 extracts a plurality of second pixel blocks 1022 from the pixel region 1023. The extracted second pixel blocks 1022 each have the same size as that of the first pixel block 1012. If the pixel region 1023 has a size of 5 pixels×5 pixels and the second pixel blocks 1022 each have a size of 3 pixels×3 pixels, nine second pixel blocks 1022 are extracted. Referring to FIG. 10, one of the extracted second pixel blocks 1022 is indicated by the hatching.

Note that the first reference image to be used by the target image 1010 is not limited to the first reference image 1020 one frame before the target image. The target image 1010 may use a plurality of X-ray images before the target image 1010, for example, an X-ray image (not shown) at time t−2 and the X-ray image 1020 at time t−1 as the first reference images.

In step S906, the similarity degree calculation unit 204 calculates the similarity degrees between the first pixel block 1012 and the respective second pixel blocks 1022. If the coordinate of a pixel 1021 placed in the center of the second pixel block 1022 are represented by a coordinate (x2, y2), the similarity degree calculation unit 204 calculates a similarity degree s(x2, y2) between the first pixel block 1012 and the second pixel block 1022 according to, for example, equation (3).

$$s ( x 2 , y 2 ) = A \times exp ( - B \times {\sum }_{{- 1 \leq i \leq 1}_{- 1 \leq j \leq 1}} \vert {I}_{t} ( x 1 + i , y 1 + j ) - {I}_{t - 1} ( x 2 + i , y 2 + j ) \vert ) \qquad ( 3 )$$

In step S907, the maximum similarity degree detection unit 802 detects the maximum value of similarity degrees s(x2, y2) calculated according to equation (4) as a maximum similarity degree S(x1, y1). The maximum similarity degree detection unit 802 gives the filter coefficient determination unit 205 the maximum similarity degree S(x1, y1) together with position information indicating the central position of the second pixel block 1022 which provides the maximum similarity degree S(x1, y1). Assume that the central position of the second pixel block 1022 which provides the maximum similarity degree S(x1, y1) is represented by a coordinate (x3, y3).

$$S ( x 1 , y 1 ) = {arg max}_{x 2 , y 2} s ( x 2 , y 2 ) \qquad ( 4 )$$

In steps S904 to S907 described above, a pixel block most similar to the first pixel block 1012 is extracted from the pixel region 1023.

In step S908, a filter coefficient determination unit 205 determines a filter coefficient G(x1, y1) based on the maximum similarity degree S(x1, y1). A method of determining the filter coefficients G(x1, y1) is the same as that in step S506, and hence a detailed description of it will be omitted. The filter coefficient storage unit 206 stores the determined filter coefficients G(x1, y1) in correspondence with position information (also called the first position information) concerning the pixel 1011 selected by the selection unit 201 and position information (also called the second position information) indicating the central position of the second pixel blocks 1022 which provides the maximum similarity degree S(x1, y1).

In step S909, the image processing unit 150 determines whether filter coefficients have been determined for all the pixels in the target image 1010. If there is any pixel for which no filter coefficient has been determined, the process returns to step S902. The image processing unit 150 repeats the processing shown in steps S902 to S908 until filter coefficients are determined for all the pixels in the target image 1010.

In step S910, a smoothing unit 209 smoothes the filter coefficient determined for each pixel. More specifically, the smoothing unit 209 generates a coefficient map (filter coefficient image) with filter coefficients being placed at pixel positions in accordance with the first position information. The smoothing unit 209 then performs smoothing processing for the coefficient map by using, for example, an averaging filter or Gaussian filter.

In step S911, a display image generation unit 207 generates a display image at time t corresponding to the target image 1010 by using the filter coefficient determined for each pixel in the target image 1010. For example, the display image generation unit 207 calculates, for each pixel, a pixel value I_{t′}(x1, y1) of the display image at time t by performing a weighted sum of the pixel value Iₜ(x1, y1) of the coordinate (x1, y1) of the target image 1010 and a pixel value I_{t-1′}(x3, y3) of the coordinate (x3, y3) of the second reference image stored in a display image storage unit 208 by using the filter coefficients G(x1, y1) according to equation (5) given below. The second reference image is a display image at time t−1 generated immediately before.

Iₜ^{′}(x3,y3)×G(x1,y1)+Iₜ(x1,y1)×(1−G(x1,y1)) (5)

As indicated by equation (5), the influence of the second reference image on the display image increases with an increase in filter coefficient. As described above, large filter coefficients are determined concerning pixels in a still region, whereas small filter coefficients G are determined concerning pixels in a dynamic region. Therefore, in the still region, the influence of the second reference image is high, and it is possible to reduce noise. In the dynamic region, the influence of the second reference image is low, and it is possible to suppress the occurrence of a residual image. This makes it possible to generate a display image with less residual image and reduced noise.

In step S911, the display image storage unit 208 temporarily stores the generated display image as the new second reference image. In step S912, the generated display image is output to a display unit 160. The display image having undergone recursive filter processing in this manner has less residual image and reduced noise. It is therefore possible to display a clear moving image without motion blur on the display unit 160.

As described above, the X-ray diagnostic apparatus including the image processing apparatus 800 according to this embodiment detects a pixel block similar to the first pixel block from the first reference image, and determines filter coefficients based on the detected pixel block, thereby generating a display image with less residual image and reduced noise. This makes it possible to display a clearer image.

Although this embodiment has exemplified the case in which the image processing unit (image processing apparatus) is incorporated in the X-ray diagnostic apparatus, the embodiment is not limited to this. The image processing apparatus may be incorporated in another apparatus such as an image display apparatus or may be implemented as an independent apparatus. In addition, the image processing apparatus is not limited to handling X-ray moving images and may be applied to any type of moving images.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus comprising:
a first storage unit configured to store data of a plurality of images;
a selection unit configured to select a pixel from a plurality of pixels included in a target image of the plurality of images;
a first extraction unit configured to extract a first pixel region including the selected pixel from the target image;
a second extraction unit configured to extract a second pixel region corresponding to the first pixel region from a reference image of the plurality of images, the reference image being different from the target image;
a calculation unit configured to calculate a similarity degree between the first pixel region and the second pixel region;
a determination unit configured to determine a filter coefficient based on the similarity degree; and
a generation unit configured to generate a display image by performing a weighted sum of the target image and a display image generated immediately before the target image, in accordance with the filter coefficient determined for each of the plurality of pixels.

2. The apparatus according to claim 1, wherein the calculation unit calculates the similarity degree based on a difference value between a pixel value of pixel in the first pixel region and a pixel value of a corresponding pixel in the second pixel region, and the determination unit increases the filter coefficient with an increase in the similarity degree.

3. The apparatus according to claim 1, wherein the second pixel region includes a pixel located at a same coordinate as a coordinate of the selected pixel.

4. The apparatus according to claim 1, further comprising:
a second storage unit configured to store a filter coefficient determined by the determination unit in correspondence with position information indicating a coordinate of the pixel selected by the selection unit; and
a smoothing unit configured to smooth a filter coefficient stored in the second storage unit in accordance with the position information,
wherein the generation unit generates a display image corresponding to the target image by performing a weighted sum of the target image and the reference image in accordance with the smoothed filter coefficient.

5. An image processing method of processing a plurality of images, comprising:
selecting a pixel from a plurality of pixels included in a target image of the plurality of images;
extracting a first pixel region including the selected pixel from the target image;
extracting a second pixel region corresponding to the first pixel region from a reference image as an image, of the plurality of images, the reference image being different from the target image;
calculating a similarity degree between the first pixel region and the second pixel region;
determining a filter coefficient based on the similarity degree; and
generating a display image by performing a weighted sum of the target image and a display image generated immediately before the target image, in accordance with the filter coefficient determined for each of the plurality of pixels.

6. An image processing apparatus comprising:
a storage unit configured to store a plurality of images;
a selection unit configured to select a pixel from a plurality of pixels included in a target image of the plurality of images and generate first position information indicating a position of the selected pixel;
a first extraction unit configured to extract a first pixel region including the selected pixel from the target image;
a setting unit configured to set a pixel region having a predetermined size on a reference image of the plurality of images, in accordance with the first position information, the reference image being different from the target image;
a second extraction unit configured to extract a plurality of second pixel regions each having a same size as a size of the first pixel region from the pixel region;
a calculation unit configured to calculate similarity degrees between the first pixel region and the plurality of second pixel regions;
a detection unit configured to detect a maximum similarity degree from the calculated similarity degrees;
a determination unit configured to determine a filter coefficient based on the maximum similarity degree; and
a generation unit configured to generate a display image by performing a weighted sum of the target image and a display image generated immediately before the target image, in accordance with a filter coefficient determined concerning each of the plurality of pixels.

7. The apparatus according to claim 6, wherein the detection unit generates second position information indicating a coordinate of a pixel included in a second pixel region which provides the maximum similarity degree, and
the generation unit generates a display image by performing a weighted sum of a pixel value of a pixel on the target image which is specified by the first position information and a pixel value of a pixel on the second reference image which is specified by the second position information, in accordance with the determined filter coefficient.

8. An image processing method of processing a plurality of images, comprising:
selecting one pixel from a plurality of pixels included in a target image of the plurality of images;
extracting a first pixel region including the selected pixel from the target image;
setting a pixel region having a predetermined size on a reference image of the plurality of images, the reference image being different from the target image;
extracting a plurality of second pixel regions each having a same size as a size of the first pixel region from the pixel region;
calculating similarity degrees between the first pixel region and the plurality of second pixel regions;
detecting a maximum similarity degree from the calculated similarity degrees;
determining a filter coefficient based on the maximum similarity degree; and
generating a display image by performing a weighted sum of the target image and a display image generated immediately before the target image, in accordance with the filter coefficient determined concerning each of the plurality of pixels.

9. An image processing apparatus comprising:
a storage unit configured to store data of a first image and data of a second image;
a calculation unit configured to calculate a plurality of similarity degrees between local regions respectively centered on a plurality of pixels included in the first image and a plurality of local regions adjacent to corresponding pixels of the second image;
a selection unit configured to select a maximum similarity degree from the plurality of similarity degrees for each pixel; and
a processing unit configured to perform a weighted sum of the first image and a display image corresponding to the second image by using weighting coefficients corresponding to the maximum similarity degrees.
